(19) United States
(12) Patent Application Publication
(10) Pub. No.: US 2024/0260837 A1
(43) Pub. Date: Aug. 8, 2024

(54) TEMPERATURE ESTIMATION SYSTEM AND TEMPERATURE ESTIMATION METHOD

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/563,148

(22) PCT Filed: Jun. 15, 2021
(86) PCT No.: PCT/JP2021/022707

§ 371 Date: Nov. 21, 2023

(51) Int. Cl. A61B 5/01; A61B 5/024; G01K 13/20

(52) U.S. Cl. CPC A61B 5/01; A61B 5/02444; G01K 13/20

(57) ABSTRACT

A temperature estimation system includes a temperature sensor for measuring a temperature of a surface of a living body, a temperature sensor for measuring a temperature inside a heat insulation material, a temperature sensor for measuring a temperature of the surface of the living body at a position remote from the temperature sensor, a learner for estimating a proportional coefficient associated with a thermal resistance of the living body on the basis of measurement results of the temperature sensors, and a temperature calculation unit for calculating a core body temperature of the living body on the basis of measurement results of the temperature sensors and the proportional coefficient

### CROSS-REFERENCE TO RELATED APPLICATIONS

This application is a national phase entry of PCT Application No. PCT/JP2021/022707, filed on Jun. 15, 2021, which application is hereby incorporated herein by reference.

### TECHNICAL FIELD

The present invention relates to a temperature estimation system and a temperature estimation method for non-invasively and accurately estimating an internal temperature of a test object such as a living body.

### BACKGROUND

It has been found from recent research on temporal biology that a circadian rhythm of a human being, that is, a so-called in-vivo clock, closely associates with various substances related to our body such as not only the quality of sleep, exercise and work, but also effects of dosing and development of diseases. The circadian rhythm is beat out substantially constant, but it is known that the circadian rhythm greatly varies depending on light with which is irradiated in a life, exercise, dietary life, age, and sex.

As an index for measuring the circadian rhythm, core body temperature is known. However, a method for measuring the core body temperature is generally a method for inserting a thermometer into the rectum or measuring the temperature of the eardrum in a state where the ear is sealed, and is a method for applying very stress as a method for measuring the core body temperature during daily activities or sleep.

On the other hand, as a technique for non-invasively measuring the core body temperature of a living body, there is a technique for estimating the core body temperature of the living body by artificially replacing the heat flow with a one-dimensional equivalent circuit model (refer to PTL 1 and NPL 1).

The method disclosed in PTL 1 and NPL 1 estimates a core body temperature T_{cbt} of a living body 100, using the thermal equivalent circuit model of the living body 100 and a sensor 101 as shown in FIG. 26. When the sensor 101 having a thermal resistance Rₛₑₙₛₒᵣ is placed on a surface of the living body 100, the core body temperature T_{cbt} of the living body 100 can be estimated using Formula (1), from a temperature Tₛₖᵢₙ on the surface side of the sensor 101 being in contact with the living body 100 and a temperature Tₜₒₚ of the upper surface of the sensor 101 on a side opposite to the surface being in contact with the living body 100.

$${T}_{cbt} = {T}_{skin} + α \times {H}_{skin} \qquad ( 1 )$$

Here, Hₛₖᵢₙ is a heat flux on the skin surface of the living body 100 and is represented by Formula (2).

$${H}_{skin} = ( {T}_{skin} - {T}_{top} ) / {R}_{skin} \qquad ( 2 )$$

In addition, a denotes a proportional coefficient associated with the thermal resistance R_{body} of the living body 100, and Rₛₖᵢₙ denotes the thermal resistance of the sensor 101.

However, in the method disclosed in PTL 1 and NPL 1, since the transportation of heat flow to the outside air through the sensor 101 from the living body 100 is assumed to be steady, when the living body 100 is exposed to wind, when the living body 100 runs, or when the living body 100 suddenly moves from a warm place to a cold place, there is a problem that a transient error occurs in estimation of the core body temperature T_{cbt}.

Also, in the conventional method, it was assumed that the thermal resistance R_{body} of the living body 100 is constant regardless of time and the proportional coefficient α is also constant. However, the blood flow state near the skin of the living body 100 varies even when the living body 100 is in the posture or exercise. Therefore, the thermal resistance R_{body} is not constant, but varies from moment to moment.

FIG. 27 shows the core body temperature T_{cbt} estimated by the conventional method and the true core body temperature (eardrum temperature) T_{ref} measured by the eardrum thermometer when a person moves back and forth between interior and exterior of the room in daily life or turns his posture sideways.

The reason of the difference between the true core body temperature T_{ref} and the estimated core body temperature T_{cbt} is that there is a difference in time until the temperature Tₜₒₚ of the upper surface of the sensor 101 and the temperature Tₛₖᵢₙ of the skin surface of the person each settle in a steady state, the blood flow varies depending on the human posture, and the like. In addition, it is not expected that the temperature will settle in a steady state in a state in which the wind is constantly changing, even though the person is rest and in a state in which the blood flow does not vary.

Further, in the conventional method, even in a state where the heat flow can be assumed to be steady, it is necessary to calibrate the proportional coefficient a one by one using another sensor such as an eardrum thermometer every time measurement is performed.

The temperature distribution in the object can be generally described by the following thermal conduction equation.

$$Equation 1$$

$$\frac{\partial T}{\partial t} = \frac{k}{c ρ} Δ T + Q \qquad ( 3 )$$

In Formula (3), T is temperature, k is thermal conductivity, c is heat capacity, ρ is density, Q is internal heat generation. Q is a term generated during exercise or the like. Δ is an operator of a double differential with respect to space, and in a three-dimensional orthogonal coordinate system, and is ∂²/∂x²+∂²/∂y²+∂²/∂z². The values k, ρ, and c related to the thermal characteristics varies from moment to moment due to various factors such as moisture content of human skin, expansion contraction of capillary vessels due to human activity, perspiration, expansion and contraction of a blood vessel due to blood pressure which changes with human posture. Since a in Formula (1) is a proportional coefficient corresponding to k/(ρc), the proportional coefficient a varies from moment to moment.

Although the conventional method disclosed in PTL 1 and NPL 1 uses the one-dimensional thermal equivalent circuit model, the one-dimensional thermal equivalent circuit model cannot be established if parameters such as the heat capacity c and the density ρ vary as described above. In other words, the internal temperature can be estimated if the heat flow is sufficiently stable, but the internal temperature cannot be estimated for an unsteady dynamic object such as a living body. For this reason, it is necessary to obtain the internal temperature (core body temperature T_{cbt}) or the proportional coefficient a in consideration of Formula (3) by some method.

### Citation List

### Patent Literature

PTL 1—Japanese Patent Application Publication No. 2020-003291

### Non Patent Literature

NPL 1—K. Kitamura et al., “Development of a new method for the noninvasive measurement of deep body temperature without a heater”, Medical Engineering & Physics, vol. 32, No. 1, pp. 1-6, 2010.

### SUMMARY

### Technical Problem

Embodiments of the present invention are for solving the problems described above, and an object thereof is to provide a temperature estimation system and a temperature estimation method capable of reducing the estimation error of the internal temperature of a test object such as a living body without calibrating a proportional coefficient every time measurement is performed.

### Solution to Problem

It is characterized in that a temperature estimation system of embodiments of the present invention includes a heat insulation material, a first temperature sensor provided on a surface of the heat insulation material facing the test object and configured to measure a temperature of a surface of the test object, a second temperature sensor configured to measure a temperature inside the heat insulation material immediately above the first temperature sensor, a third temperature sensor configured to measure a temperature of a surface of the test object at a position remote from the first temperature sensor, a learner configured to estimate a proportional coefficient associated with a thermal resistance of the test object on the basis of measurement results of the first, second and third temperature sensors, and a temperature calculation unit configured to calculate an internal temperature of the test object on the basis of measurement results of the first and second temperature sensors and the proportional coefficient.

In addition, one configuration example of the temperature estimation system according to embodiments of the present invention further includes a heart rate measurement unit configured to measure a heart rate of the test object, and it is characterized in that the learner estimates the proportional coefficient on the basis of measurement results of the first, second, and third temperature sensors and measurement result of the heart rate measurement unit.

Also, it is characterized in that the temperature estimation system includes a heat insulation material, a first temperature sensor provided on a surface of the heat insulation material facing a test object and configured to measure a temperature of a surface of the test object, a second temperature sensor configured to measure a temperature inside the heat insulation material immediately above the first temperature sensor, a third temperature sensor configured to measure a temperature of a surface of the test object at a position remote from the first temperature sensor, and a learner configured to estimate an internal temperature of the test object on the basis of measurement results of the first, second, and third temperature sensors.

In addition, one configuration example of the temperature estimation system according to embodiments of the present invention further includes a heart rate measurement unit configured to measure a heart rate of the test object, and it is characterized in that the learner estimates the internal temperature of the test object on the basis of measurement results of the first, second, and third temperature sensors and measurement result of the heart rate measurement unit.

Further, one configuration example of the temperature estimation system of embodiments of the present invention includes a plurality of the learners prepared in advance for coping with a state of the test object or an environment around the test object, and it is characterized in that the temperature estimation system includes a selection unit configured to select a learner corresponding to the state of the test object or the environment around the test object for the estimation from among the plurality of learners on the basis of at least one of a measurement result of the first temperature sensor and a measurement result of the second temperature sensor.

In addition, one configuration example of the temperature estimation system of embodiments of the present invention includes a plurality of the learners prepared in advance for coping with a state of the test object or an environment around the test object and it is characterized in that the temperature estimation system further includes a selection unit configured to select a learner corresponding to the state of the test object or the environment around the test object for the estimation from among the plurality of learners on the basis of at least one of a measurement result of the first temperature sensor, a measurement result of the second temperature sensor, and a measurement result of the heart rate measurement unit.

Furthermore, it is characterized in that a temperature estimation method includes a first step of measuring a temperature of a surface of a test object by a first temperature sensor provided on a surface of a heat insulation material facing the test object, a second step of measuring a temperature inside the heat insulation material immediately above the first temperature sensor by a second temperature sensor, a third step of measuring a temperature of a surface of the test object remote from the first temperature sensor by a third temperature sensor, a fourth step of estimating a proportional coefficient associated with a thermal resistance of the test object by a learned learner on the basis of measurement results of the first, second, and third steps, and a fifth step of calculating an internal temperature of the test object on the basis of measurement results of the first and second steps and the proportional coefficient.

In addition, one configuration example of the temperature estimation method of embodiments of the present invention further includes a sixth step of measuring a heart rate of the test object, and it is characterized in that the fourth step includes a step of estimating the proportional coefficient on the basis of measurement results of the first, second, and third steps and a measurement result of the sixth step.

### Advantageous Effects of Embodiments of Invention

According to embodiments of the present invention, a proportional coefficient is estimated on the basis of measurement results of first, second, and third temperature sensors or measurement results of the first, second, and third temperature sensors and measurement result of a heart rate measurement unit, and an internal temperature of a test object is calculated, so that the internal temperature of the test object can be accurately estimated without calibrating the proportional coefficient every time measurement is performed.

In addition, in embodiments of the present invention, on the basis of the measurement results of the first, second, and third temperature sensors, or the measurement results of the first, second, and third temperature sensors and the measurement result of the heart rate measurement unit, the internal temperature of the test object is estimated, so that the internal temperature of the test object can be accurately estimated without calibrating the proportional coefficient every time measurement is performed.

### BRIEF DESCRIPTION OF THE DRAWINGS

FIG. 1 is a schematic diagram showing an arrangement of sensors on a surface of a living body.

FIG. 2 is a block diagram showing a configuration of a temperature estimation system according to a first example of the present invention.

FIG. 3 is a plan view showing a housing surface of a temperature measurement unit shown in FIG. 2.

FIG. 4 is a block diagram showing another example of the temperature measurement unit according to the first example of the present invention.

FIG. 5 is a plan view showing a housing surface of the temperature measurement unit shown in FIG. 4.

FIG. 6 is a block diagram showing another example of the temperature measurement unit according to the first example of the present invention.

FIG. 7 is a block diagram showing another example of the temperature measurement unit according to the first example of the present invention.

FIG. 8 is a block diagram showing another example of the temperature measurement unit according to the first example of the present invention.

FIG. 9 is a plan view showing a housing surface of the temperature measurement unit shown in FIG. 8.

FIG. 10 is a block diagram showing another example of the temperature measurement unit according to the first example of the present invention.

FIG. 11 is a block diagram showing another example of the temperature measurement unit according to the first example of the present invention.

FIG. 12 is a plan view showing a housing surface of the temperature measurement unit shown in FIG. 11.

FIG. 13 is a plan view showing another arrangement example of temperature sensors according to the first example of the present invention.

FIG. 14 is a plan view showing another arrangement example of temperature sensors according to the first example of the present invention.

FIG. 15 is a block diagram showing another example of the temperature measurement unit according to the first example of the present invention.

FIG. 16 is a flowchart explaining operations of the temperature estimation system according to the first example of the present invention.

FIG. 17 is a diagram explaining a tensor generation method according to the first example of the present invention.

FIG. 18 is a flowchart explaining another operations of the temperature estimation system according to the first example of the present invention.

FIG. 19A is a diagram showing an example of core body temperature estimated by the method of the first example of the present invention.

FIG. 19B is a diagram showing an example of true core body temperature measured by an eardrum thermometer.

FIG. 20 is a block diagram showing a configuration of a server device of the temperature estimation system according to a second example of the present invention.

FIG. 21 is a flowchart explaining operations of the temperature estimation system according to the second example of the present invention.

FIG. 22 is a flowchart explaining another operation of the temperature estimation system according to the first example of the present invention.

FIG. 23 is a flowchart explaining another operation of the temperature estimation system according to the first example of the present invention.

FIG. 24 is a flowchart explaining another operation of the temperature estimation system according to the second example of the present invention.

FIG. 25 is a block diagram showing a configuration example of a computer that implements the temperature estimation system according to the first and second examples of the present invention.

FIG. 26 is a diagram showing the thermal equivalent circuit model of a living body and a sensor.

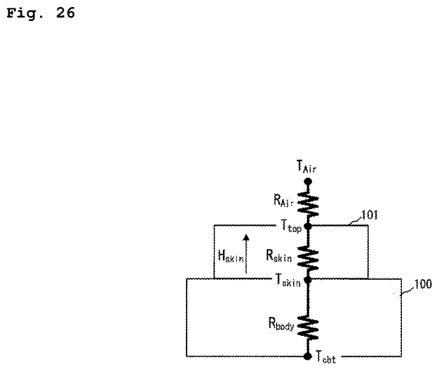

FIG. 27 is a diagram showing examples of the core body temperature estimated by the conventional method and the true core body temperature measured by an eardrum thermometer.

### DETAILED DESCRIPTION OF ILLUSTRATIVE EMBODIMENTS

### Principles of Embodiments of the Invention

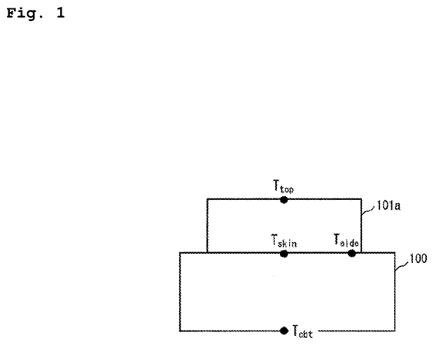

If values corresponding to ∂T/∂t, ΔT, and Q in Formula (3) can be measured, k/(cρ) in Formula (3) can be estimated. FIG. 1 shows a schematic diagram in which a sensor 101a is arranged on a surface of a living body 100. The heat conduction equation shown by Formula (3) is also established in the living body 100 near the sensor 101a.

The values k, ρ, and c related to the thermal characteristics are parameters which change from moment to moment, but the changes of the spatial characteristics of k, ρ, and c have features to be relatively small. That is, it is assumed that the values of k, ρ, and c change from moment to moment, but are substantially uniform in terms of space. It can be said that the thermal conduction equation of Formula (3) in the vicinity of the sensor 101a is also associated with to the core body temperature T_{cbt}. In addition, since a proportional coefficient a associated with the thermal resistance of the living body 100 corresponds to k/(cρ), it is associated with the thermal conduction equation of Formula (3) in the vicinity of the sensor 101a.

Therefore, the core body temperature T_{cbt} can be estimated on the basis of the relation of Formula (3) in the vicinity of the sensor 101a. Further, the core body temperature T_{cbt} can be estimated from Formula (1) by using the proportional coefficient a estimated based on Formula (3).

Here, considering the relation of Formula (3) in the vicinity of the sensor 101a, in order to estimate ∂T/∂t, ΔT, it is necessary to measure time series data of temperature, temperature distribution on the surface of the living body 100, and heat flowing from the surface of the living body 100 to the sensor 101a. For example, as shown in FIG. 1, when the temperature T_{side} of the skin surface at a position remote from Tₛₖᵢₙ is measured in addition to the conventional temperatures Tₛₖᵢₙ and Tₜₒₚ, a value corresponding to ΔT can be obtained from the difference between Tₛₖᵢₙ and T_{side} and the difference between Tₛₖᵢₙ and Tₜₒₚ. On the other hand, Q is closely related to the intensity of the exercise of the living body 100, so that it can be analogized from a heart rate N and activity amount A of the living body 100.

That is, by a function f indicating a relationship between the temperature Tₛₖᵢₙ, Tₜₒₚ, T_{side}, and the heart rate N, and the core body temperature T_{cbt}, the core body temperature Tcbtcan be estimated.

$${T}_{cbt} = f ( {T}_{skin} , {T}_{top} , {T}_{side} , N ) \qquad ( 4 )$$

Further, by using a function g indicating a relationship between the temperature Tₛₖᵢₙ, Tₜₒₚ, T_{side}, and the heart rate N, and the proportional coefficient a, the proportional coefficient a can be estimated.

$$α = g ( {T}_{skin} , {T}_{top} , {T}_{side} , N ) \qquad ( 5 )$$

In general, it is difficult to associate Formula (3) with Tₛₖᵢₙ, Tₜₒₚ, T_{side}, and N by an elementary function, but it is possible to associate by using a non-dimensional number or a convolution neural network.

### FIRST EXAMPLE

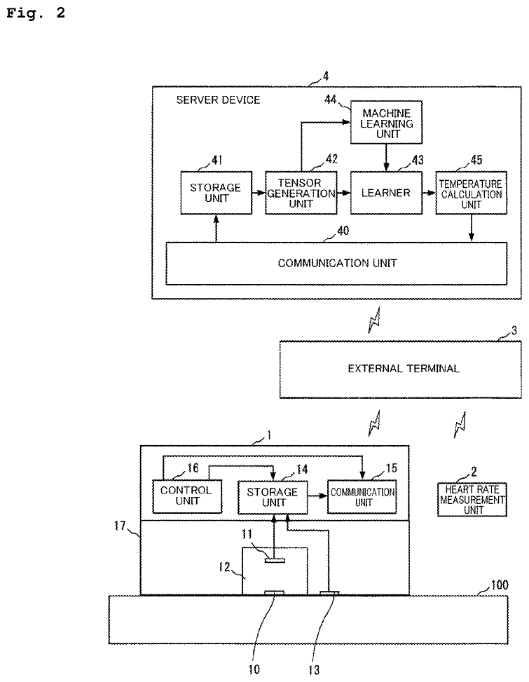

Example of embodiments of the present invention will be described below with reference to the accompanying drawings. FIG. 2 is a block diagram showing a configuration of a temperature estimation system according to a first example of the present invention. The temperature estimation system includes a temperature measurement unit 1 (temperature measurement device), a heart rate measurement unit 2 (heart rate measurement device), a terminal 3 such as a PC (Personal Computer), a smart phone, or the like, and a server device 4.

The temperature measurement unit 1 includes a temperature sensor 10 for measuring a temperature Tₛₖᵢₙ of a skin surface of a living body 100 (a test object such as a human body), a temperature sensor 11 for measuring a temperature Tₜₒₚ inside a heat insulation material 12 immediately above the temperature sensor 10, a heat insulation material 12 for holding the temperature sensor 10 and the temperature sensor 11, a temperature sensor 13 for measuring a temperature T_{side} of the skin surface of the living body 100 remote from the temperature sensor 10, a storage unit 14 for storing data, a communication unit 15 for transmitting the data of the temperatures Tₛₖᵢₙ, Tₜₒₚ, and T_{side} to the terminal 3, and a control unit 16 for controlling the reading/writing and the communication of the data to/from the storage unit 14.

The temperature measurement unit 1 is arranged so that, for example, the surface of the resin housing 17 and the heat insulation material 12 exposed on this surface come into contact with the skin of the living body 100. The temperature sensor 10 is provided on a living body side surface of the heat insulation material 12. The temperature sensor 11 is provided inside the heat insulation material 12 immediately above the temperature sensor 10. The heat insulation material 12 holds the temperature sensor 10 and the temperature sensor 11, and serves as a resistor against heat flowing into the temperature sensor 11. As a material of the heat insulation material 12, for example, PET resin is used. In addition, the temperature sensor 13 is arranged at a position remote from the temperature sensor 10 so as to come into contact with the skin of the living body 100.

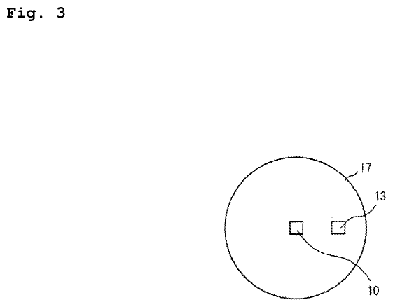

FIG. 3 is a plan view showing the surface of the housing 17 of the temperature measurement unit 1 in contact with the living body 100. As shown in FIG. 3, the temperature sensor 13 is arranged at one location around the temperature sensor 10. As will be described later, a plurality of temperature sensors 13 may be arranged around the temperature sensor 10.

The heart rate measurement unit 2 measures the heart rate N of the living body 100 by, for example, a photoplethysmogram. As an example of the heart rate measurement unit 2, there is, for example, a wristwatch-type heart rate measurement device.

The server device 4 includes a communication unit 40 for transmitting and receiving data to and from the terminal 3, a storage unit 41 for storing data, a tensor generation unit 42 for converting the temperature Tₛₖᵢₙ, T_{side}, Tₜₒₚ and the heart rate N into tensors, a learned learner 43 for estimating the proportional coefficient a for the temperature Tₛₖᵢₙ, T_{side}, and Tₜₒₚ or the proportional coefficient a for the temperature Tₛₖᵢₙ, T_{side}, Tₜₒₚ, and the heart rate N, a machine learning unit 44 for performing machine learning of the learner 43, and a temperature calculation unit 45 for calculating the core body temperature T_{cbt} (internal temperature) of the living body 100.

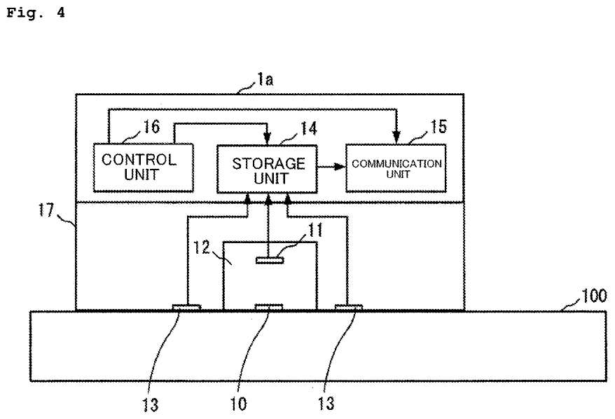

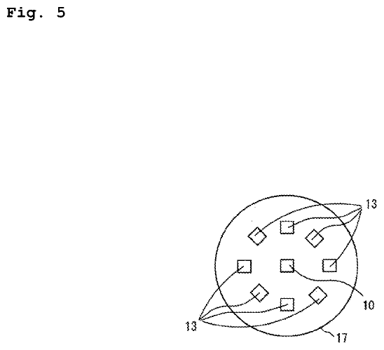

FIG. 4 is a diagram showing another example of the temperature measurement unit. The temperature measurement unit 1a of FIG. 4 includes a plurality of temperature sensors 13. FIG. 5 is a plan view showing the surface of the housing 17 of the temperature measurement unit 1a in contact with the living body 100. As shown in FIG. 5, a plurality of temperature sensors 13 is arranged around the temperature sensor 10 on the surface of the housing 17 of the temperature measurement unit 1a.

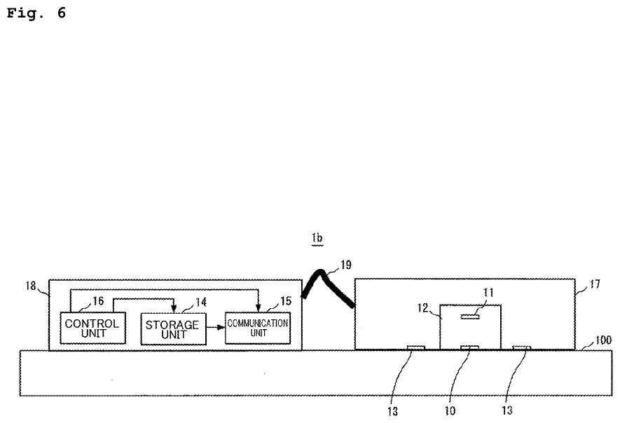

FIG. 6 is a diagram showing another example of the temperature measurement unit. In the temperature measurement unit 1b shown in FIG. 6, the housing 17 for accommodating the temperature sensors 10 and 13 and the heat insulation material 12 and the housing 18 for accommodating the storage unit 14, the communication unit 15, and the control unit 16 are separated. The temperature sensors 10 and 13 are connected to a device on the housing 18 side by wiring 19. The arrangement of the temperature sensors 10 and 13 as viewed from the living body 100 side is the same as that shown in FIG. 5.

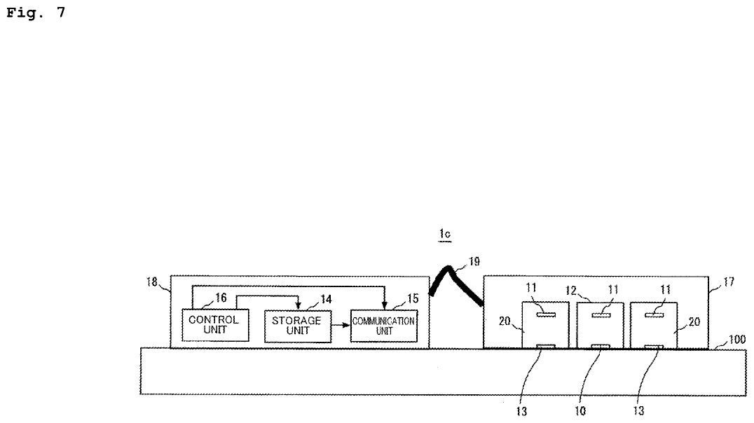

FIG. 7 is a diagram showing another example of the temperature measurement unit. In the temperature measurement unit 1c of FIG. 7, a plurality of heat insulation materials 20 is arranged around the heat insulation material 12 in the temperature measurement unit 1b shown in FIG. 6. The temperature sensor 10 is provided on a living body side surface of the heat insulation material 12. The temperature sensor 13 is provided on a living body side surface of the heat insulation material 20. The temperature sensor 11 is provided inside the heat insulation materials 12 and 20 immediately above the temperature sensors 10 and 13. The arrangement of the temperature sensors 10 and 13 as viewed from the living body 100 side is the same as that shown in FIG. 5.

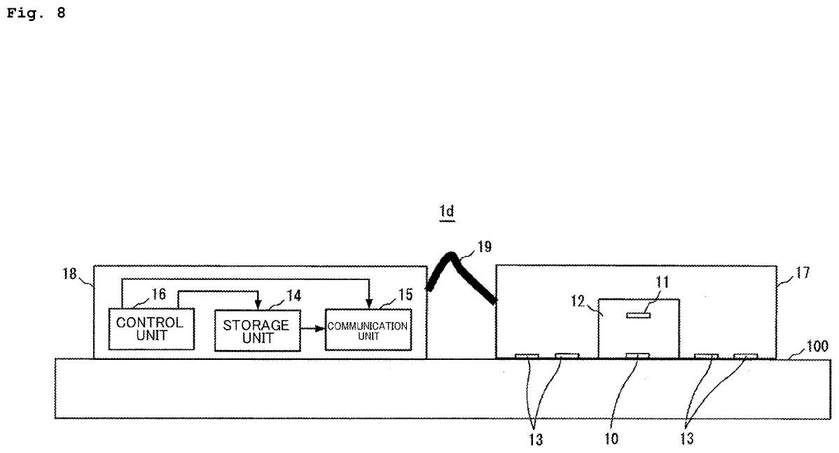

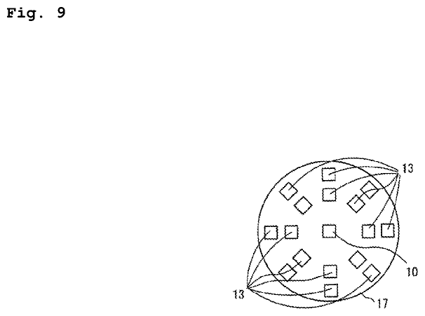

FIG. 8 is a diagram showing another example of the temperature measurement unit. In the temperature measurement unit id of FIG. 8, the temperature sensor 13 is doubly arranged around the temperature sensor 10 in the temperature measurement unit 1b shown in FIG. 6. FIG. 9 is a plan view showing the surface of the housing 17 of the temperature measurement unit 1d in contact with the living body 100.

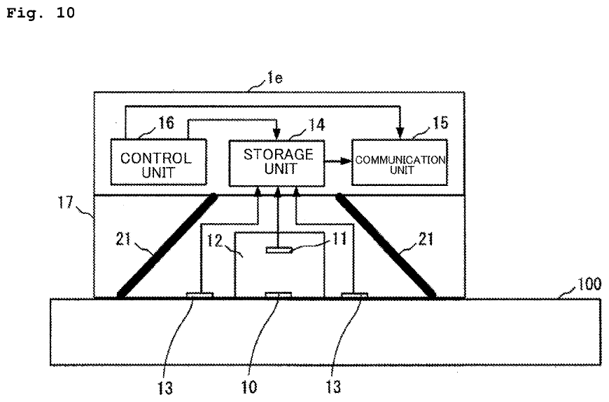

FIG. 10 is a diagram showing another example of the temperature measurement unit. In the temperature measurement unit 1e of FIG. 10, an internal structure 21 for suppressing the influence of outside air and wind is provided in a housing 17 in the temperature measurement unit 1a shown in FIG. 4. The temperature sensors 10, 11, and 13 are shut off from the outside air by the internal structure 21. The internal structure 21 is made of a material having a good thermal conductivity such as aluminum. The internal structure 21 has, for example, a truncated cone shape or a dome shape, and covers the temperature sensors 10, 11, and 13 and the heat insulation material 12. The arrangement of the temperature sensors 10 and 13 as viewed from the living body 100 side is the same as that shown in FIG. 5.

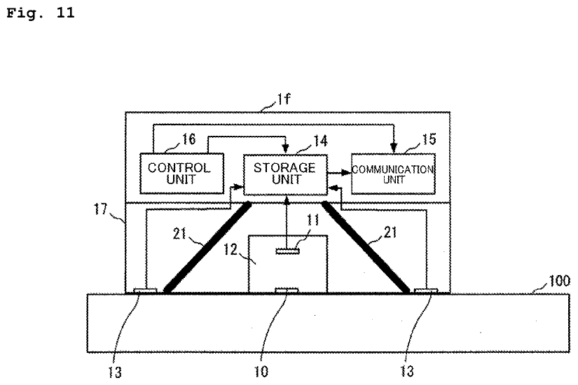

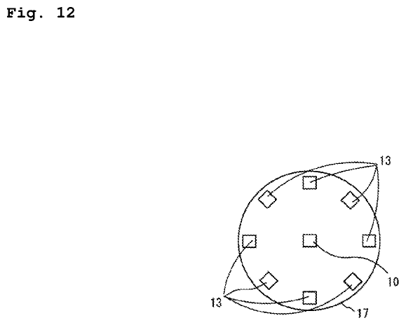

FIG. 11 is a diagram showing another example of the temperature measurement unit. The temperature measurement unit if shown in FIG. 11 has the same constitution as that of the temperature measurement unit 1e shown in FIG. 10. However, the temperature sensor 13 is arranged not inside the internal structure 21 but outside the internal structure 21. FIG. 12 is a plan view showing the surface of the housing 17 of the temperature measurement unit if in contact with the living body 100.

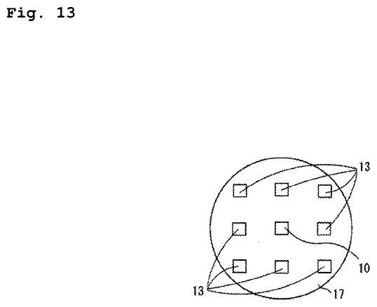

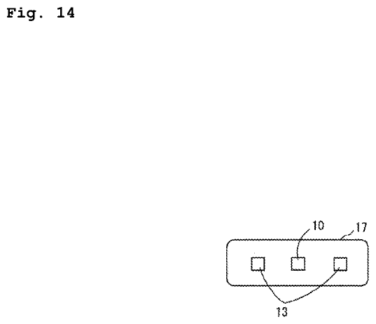

In addition, in the temperature measurement units 1a, 1b, 1c, 1e, and 1f, the temperature sensor 13 may be arranged as shown in FIG. 13. Further, in the temperature measurement units 1a, 1b, 1c, 1e, and 1f, the temperature sensor 13 may be arranged as shown in FIG. 14.

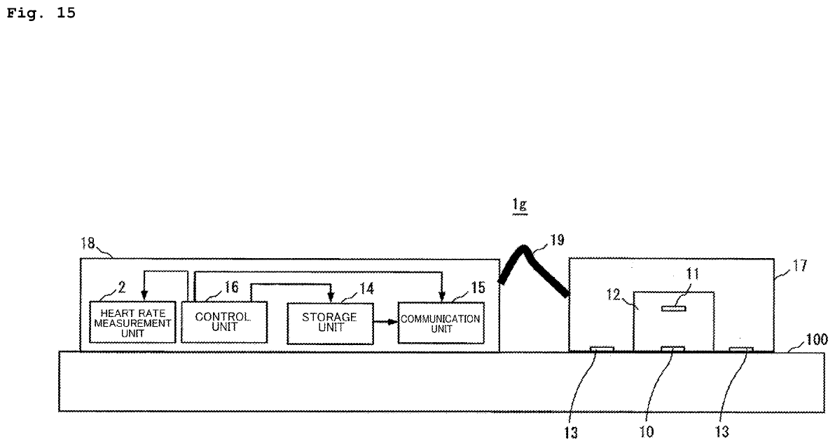

FIG. 15 is a diagram showing another example of the temperature measurement unit. The temperature measurement unit 1g shown in FIG. 15 incorporates the heart rate measurement unit 2 therein. The arrangement of the temperature sensors 10 and 13 as viewed from the living body 100 side is the same as that shown in FIG. 5.

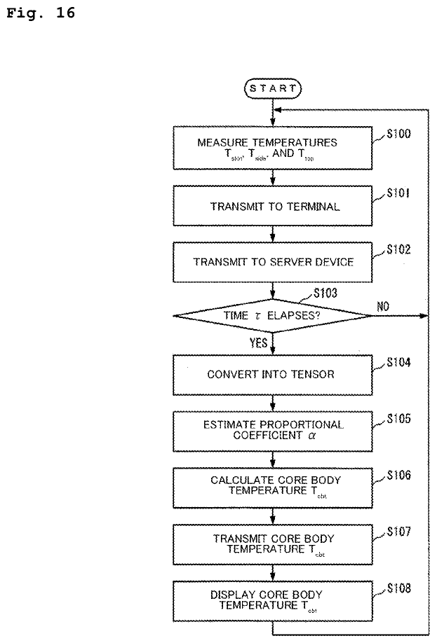

FIG. 16 is a flowchart explaining operations of the temperature estimation system of the present example. Note that, in FIG. 16, an example in which the heart rate measurement unit 2 is not used will be described.

The temperature sensor 10 of the temperature measurement unit 1 and 1a to 1g measures the temperature Tₛₖᵢₙ of the skin surface of the living body 100. The temperature sensor 13 measures the temperature T_{side} of the skin surface of the living body 100 at a position remote from the temperature sensor 10. The temperature sensor 11 measures a temperature Tₜₒₚof the inside of the heat insulation material 12 at a position away from the living body 100 (step S100 shown in FIG. 16). Measured data of the temperature sensors 10, 13, and 11 is stored in the storage unit 14 once. The temperature sensors 10, 13, and 11 measure the temperatures Tₛₖᵢₙ, T_{side} and Tₜₒₚ at fixed intervals, for example, every second.

The communication units 15 of the temperature measurement units 1 and 1a to 1g transmits data of the temperatures Tₛₖᵢₙ, T_{side}, and Tₜₒₚ to the terminal 3 such as a PC, a smart phone, or the like (step S101 shown in FIG. 16).

The terminal 3 transmits the received data of the temperatures Tₛₖᵢₙ, T_{side}, and Tₜₒₚ to the server device 4 (step S102 shown in FIG. 16). The communication unit 40 of the server device 4 stores the received data of the temperatures Tₛₖᵢₙ, T_{side}, and Tₜₒₚ in the storage unit 41.

Next, the tensor generation unit 42 of the server device 4 converts each time series data of the temperature Tₛₖᵢₙ, T_{side}, and Tₜₒₚ for time τ into a tensor (step S104 shown in FIG. 16) after a necessary time t (for example, 1024 seconds) for acquiring the time series data required for the dataset used in a deep learning elapses (YES in step S103 shown in FIG. 16).

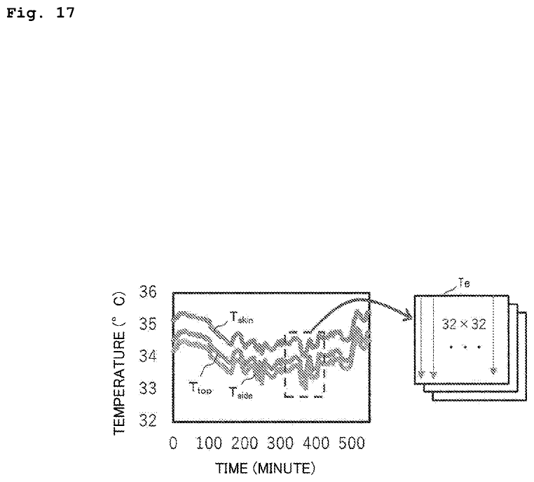

For example, it is assumed that there is time series data of the temperatures Tₛₖᵢₙ, T_{side}, and Tₜₒₚ measured every second as shown in FIG. 17. When converting each time series data for 1024 seconds of the temperatures Tₛₖᵢₙ, T_{side}, and Tₜₒₚ into the tensor, the tensor generation unit 42 subtracts 32° C. from Tₛₖᵢₙ, T_{side}, and Tₜₒₚ, respectively, further divides the data after the subtraction by 4° C., and normalizes the temperatures Tₛₖᵢₙ, T_{side}, and Tₜₒₚ, respectively. Then, as shown in the example of the tensor Te shown in FIG. 17, the data of the respective times of the normalized temperatures Tₛₖᵢₙ, T_{side}, and Tₜₒₚ may be arranged as pixels of the tensor (image).

Note that, in the case where the temperature Tₜₒₚ is measured by the plurality of temperature sensors 11, each temperature Tₜₒₚ may be normalized, and data of each time of the normalized temperature Tₜₒₚ may be arranged as pixels of the tensor. Similarly, in the case where the temperature T_{side} is measured by the plurality of temperature sensors 13, the respective temperature T_{side} may be normalized, and the data of the respective time of the normalized temperature T_{side} may be arranged as pixels of the tensor.

Next, the learner 43 of the server device 4 is a model configured with software in which the relationship between the temperatures Tₛₖᵢₙ, T_{side}, and Tₜₒₚ and the proportional coefficient a or the relationship between the temperatures Tₛₖᵢₙ, T_{side}, and Tₜₒₚ, the heart rate N, and the proportional coefficient α is modeled. The learner 43 outputs an estimation result of the proportional coefficient α when the tensor is inputted from the tensor generation unit 42 (step S105 shown in FIG. 16). As an example of the learner 43, for example, a CNN (Convolutional Neural Network) learner is known.

The learner 43 must be made to learn in advance. Specifically, each time series data of the temperature Tₛₖᵢₙ, T_{side}, and Tₜₒₚ of the living body 100 is acquired at the time of a prior test, and, for example, the time series data of the true core body temperature T_{ref} is acquired by the eardrum thermometer at the same time. The proportional coefficient a is calculated by Formula (6) at each time point by using the data of the temperatures Tₛₖᵢₙ, Tₜₒₚ, and T_{ref} at the same time point, thereby obtaining time series data of the proportional coefficient a.

$$α = ( {T}_{ref} - {T}_{skin} ) / ( {T}_{skin} - {T}_{top} ) \qquad ( 6 )$$

Then, each time series data of the temperatures Tₛₖᵢₙ, T_{side}, and Tₜₒₚ and the proportional coefficient a is converted into the tensor by the tensor generation unit 42. The machine learning unit 44 of the server device 4 performs machine learning of the learner 43 by using the tensor. Specifically, the machine learning unit 44 sets each time series data of the temperatures Tₛₖᵢₙ, T_{side}, and Tₜₒₚ as an input variable of the learner 43 and the proportional coefficient a as an output variable of the learner 43, and perform the machine learning of the learner 43 so as to obtain a target output variable. Thus, the learner 43 can be made to learn before the estimation of the proportional coefficient α in the step S105.

Next, the temperature calculation unit 45 of the server device 4 calculates a heat flux Hₛₖᵢₙ on the skin surface of the living body 100 by Formula (2) on the basis of the temperatures Tₛₖᵢₙ and Tₜₒₚ, and calculates the core body temperature T_{cbt} of the living body 100 by Formula (1) on the basis of the temperature Tₛₖᵢₙ, the heat flux Hₛₖᵢₙ, and the proportional coefficient α estimated by the learner 43 (step S106 shown in FIG. 16). The thermal resistance Rₛₖᵢₙ of the heat insulation material 12 is stored in advance in the storage unit 41. Note that the temperature calculation unit 45 may calculate the core body temperature T_{cbt} by Formula (7) without using the heat flux Hₛₖᵢₙ.

$${T}_{cbt} = {T}_{skin} + α \times ( {T}_{skin} - {T}_{top} ) \qquad ( 7 )$$

As for the temperatures Tₛₖᵢₙ and Tₜₒₚ used for calculating the core body temperature T_{cbt}, the core body temperature T_{cbt} may be calculated by using the respective latest values of the time series data of the temperatures Tₛₖᵢₙ and Tₜₒₚ for the time t or representative values (for example, average values) of each of the temperatures Tₛₖᵢₙ and Tₜₒₚ of the time t may be used. In addition, when the temperature Tₜₒₚ is measured by the plurality of temperature sensors 11, the core body temperature T_{cbt} may be calculated by using the temperature Tₜₒₚ measured by the temperature sensor 11 at one predetermined location, or a representative value (for example, an average value) of the temperature Tₜₒₚ measured by the plurality of temperature sensors 11 may be used.

In this example, it is sufficient that the core body temperature T_{cbt} is measured to about the first decimal point in the range of 30° C. to 42.0° C. for the temperature accuracy ±0.1° C. For this reason, when a CNN learner is used, for example, 120 output layers of the learner 43 may be prepared.

When learning by the learner 43 is not sufficient or data of overlearning is included, the estimated value of the core body temperature T_{cbt} may be extremely large or small. The probability of occurrence of such an abnormal value of the core body temperature T_{cbt} is small. Therefore, the abnormal value of the core body temperature T_{cbt} can be sufficiently removed by statistical signal processing such as a particle filter and a filter of a first-order lag system used in classical control.

The communication unit 40 of the server device 4 transmits data of the core body temperature T_{cbt} calculated by the temperature calculation unit 45 to the terminal 3 (step S107 shown in FIG. 16).

The terminal 3 displays the value of the core body temperature T_{cbt} received from the server device 4 (step S108 shown in FIG. 16).

As described above, the core body temperature T_{cbt} is calculated for each time t. In the present example, the estimation error of the core body temperature T_{cbt} can be reduced.

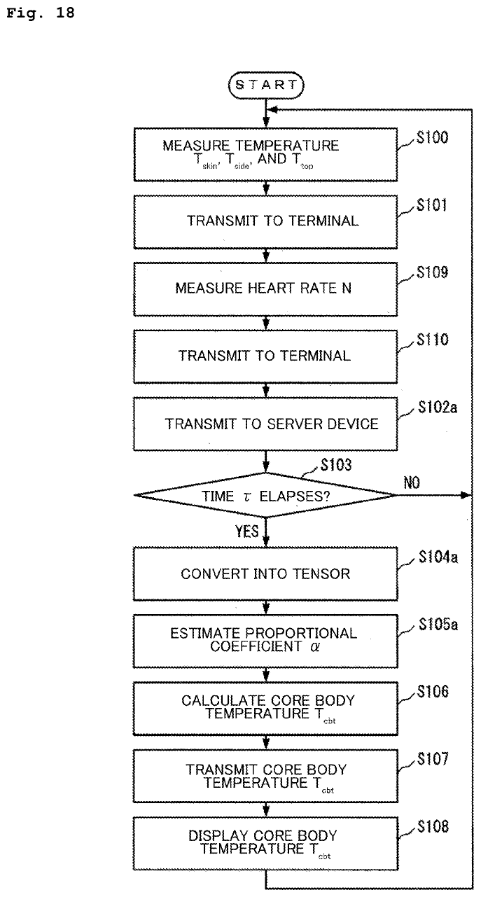

Note that when the exercise intensity of the living body 100 is large, the heart rate N of the living body 100 is preferably acquired. The operations of the temperature estimation system in this case will be described with reference to FIG. 18. The processing of steps S100 and S101 is as described above.

The heart rate measurement unit 2 measures the heart rate N (instantaneous heart rate) of the living body 100 at fixed intervals, for example, every second (step S109 shown in FIG. 18). The heart rate measurement unit 2 transmits data of the heart rate N to the terminal 3 (step S110 shown in FIG. 18). Note that when the heart rate measurement unit 2 is provided inside the temperature measurement unit 1g as shown in FIG. 15, the data of the heart rate N is transmitted from the communication unit 15 to the terminal 3.

The terminal 3 transmits the received data of the temperatures Tₛₖᵢₙ, T_{side}, and Tₜₒₚ and data of the heart rate N to the server device 4 (step S102a shown in FIG. 18). The communication unit 40 of the server device 4 stores the received data of the temperatures Tₛₖᵢₙ, T_{side}, and Tₜₒₚ and data of the heart rate N in the storage unit 41.

The tensor generation unit 42 of the server device 4 converts each time series data of the temperatures Tₛₖᵢₙ, T_{side}, and Tₜₒₚ for the time t and the heart rate N for the time t into the tensor (step S104a shown in FIG. 18) after the lapse of the time t (YES in step S103 shown in FIG. 18). In this case, the data of the heart rate N may be arranged as pixels of the tensor, similarly to the temperatures Tₛₖᵢₙ, T_{side}, and Tₜₒₚ.

The learner 43 of the server device 4 outputs an estimation result of the proportional coefficient a when the tensor is inputted from the tensor generation unit 42 (step S105a shown in FIG. 18). When the data of the heart rate N is used, each time series data of the temperatures Tₛₖᵢₙ, T_{side}, and Tₜₒₚ and the heart rate N of the living body 100 is acquired in a prior test, and time series data of true core body temperature T_{ref} is acquired by the eardrum thermometer at the same time. The proportional coefficient a is calculated by Formula (6) at each time point by using the data of the temperatures Tₛₖᵢₙ, Tₜₒₚ, and T_{ref} at the same time, thereby obtaining time series data of the proportional coefficient α.

Then, each time series data of the temperatures Tₛₖᵢₙ, T_{side}, and Tₜₒₚ, the heart rate N, and the proportional coefficient a is converted into the tensor by the tensor generation unit 42. The machine learning unit 44 of the server device 4 sets each time series data of temperatures Tₛₖᵢₙ, T_{side}, and Tₜₒₚ and time series data of heart rate N as an input variable of the learner 43, sets a proportional coefficient a as an output variable of the learner 43, and performs the machine learning of the learner 43 so as to obtain a target output variable. Thus, the learner 43 can be made to learn before the estimation of the proportional coefficient a in the step S105a.

The processing of steps S106 to S108 is as described above. Thus, by using the data of the heart rate N, the estimation error of the core body temperature T_{cbt} can be reduced even when the exercise intensity of the living body 100 is large.

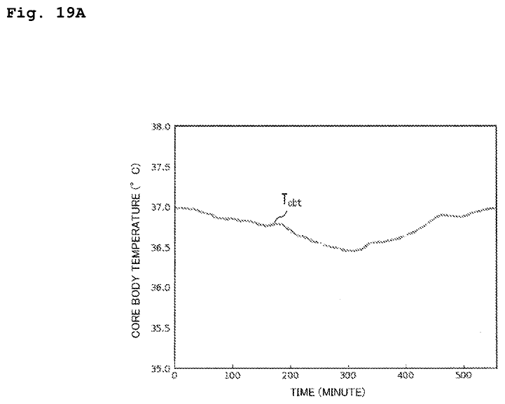

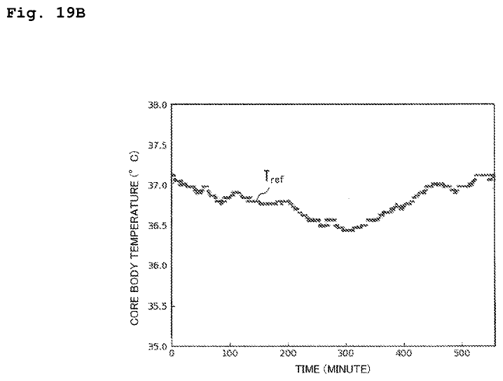

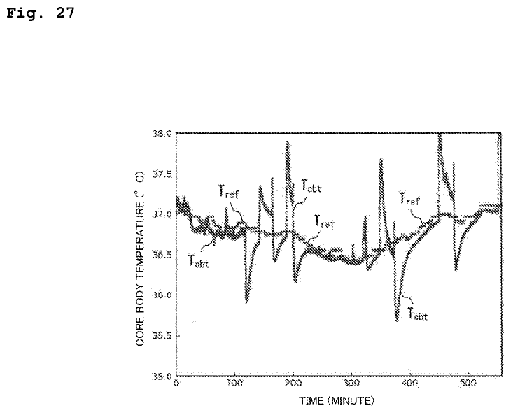

FIG. 19A shows the core body temperature T_{cbt} estimated by the method of the present example when a person moves back and forth between interior and exterior of the room in daily life or turns his posture sideways, and FIG. 19B shows the true core body temperature (eardrum temperature) T_{ref} measured by the eardrum thermometer. As compared with the core body temperature T_{cbt} estimated by the conventional method shown in FIG. 27, according to the present example, noise is suppressed and the error is corrected, and it can be found that the estimation result close to the eardrum temperature is obtained. According to the present example, it is not necessary to calibrate the proportional coefficient a every measurement, and it is possible to accurately estimate the core body temperature T_{cbt} without being affected by the blood flow of the living body or by the outside air temperature or wind.

### SECOND EXAMPLE

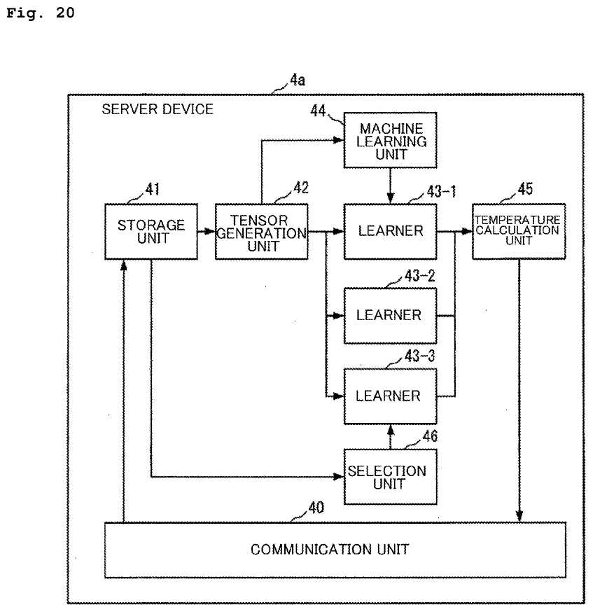

Next, a description will be given of a second example of the present invention. FIG. 20 is a block diagram showing a configuration of a server device of a temperature estimation system according to the second example of the present invention. A server device 4a of the present example includes a communication unit 40, a storage unit 41, a tensor generation unit 42, a plurality of learners 43-1 to 43-3 prepared in advance for coping with the state of the living body 100 or the environment around the living body 100, a machine learning unit 44, a temperature calculation unit 45, and a selection unit 46 for selecting a learner corresponding to the state of the living body 100 or the surrounding environment from among the plurality of learners 43-1 to 43-3 on the basis of at least one of the temperatures Tₛₖᵢₙ and Tₜₒₚ and the heart rate N.

The configurations of the temperature measurement units 1 and 1a to 1g are as described in the first example.

The accuracy of the proportional coefficient a can be determined with respect to the required temperature accuracy. In the present example, it is sufficient that the proportional coefficient a which varies in the state of blood flow or the like with respect to the temperature accuracy ±0.1° C. is measured to about 2.0 to 12.0 and to about the first decimal point. Therefore, when a CNN learner is used, for example, if 100 output layers of the learner are prepared, it is possible to cope with the change and the accuracy of the proportional coefficient α. When the range of a can be limited to some extent, there is an advantage that the output layer can be made smaller than the case where the core body temperature is estimated as it is.

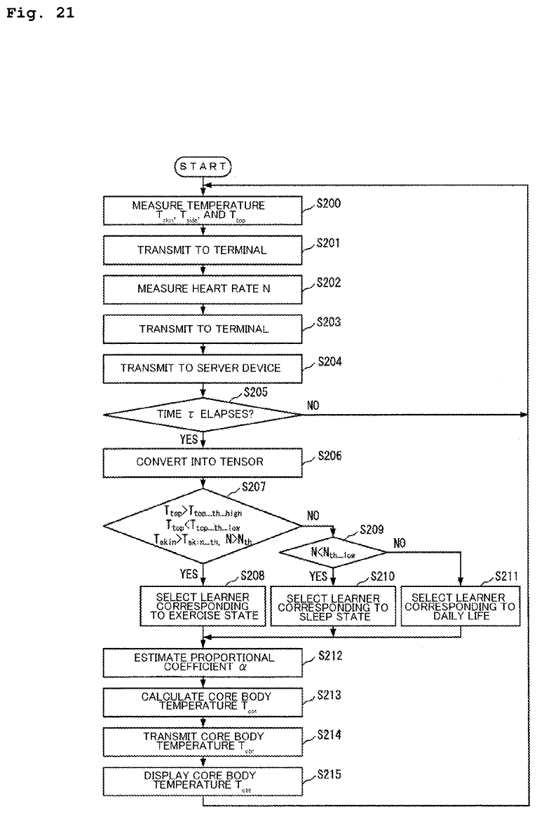

FIG. 21 is a flowchart showing operations of the temperature estimation system of the present example. Similarly to the first example, the temperature sensors 10, 13, and 11 of the temperature measurement units 1 and 1a to 1g measure the temperatures Tₛₖᵢₙ, T_{side}, and Tₜₒₚ, for example, every second (step S200 in shown FIG. 21).

The communication units 15 of the temperature measurement units 1 and 1a to 1g transmit data of the temperatures Tₛₖᵢₙ, T_{side}, and Tₜₒₚ to the terminal 3 (step S201 shown in FIG. 21).

The heart rate measurement unit 2 measures the heart rate N (instantaneous heart rate) of the living body 100, for example, every second (step S202 shown in FIG. 21). The heart rate measurement unit 2 transmits data of the heart rate N to the terminal 3 (step S203 shown in FIG. 21). As shown in FIG. 15, when the heart rate measurement unit 2 is provided inside the temperature measurement unit 1g, data of the heart rate N is transmitted from the communication unit 15 to the terminal 3.

The terminal 3 transmits the received data of the temperatures Tₛₖᵢₙ, T_{side}, and Tₜₒₚ and the data of the heart rate N to the server device 4a (step S204 shown in FIG. 21).

The tensor generation unit 42 of the server device 4a converts each time series data of the temperatures Tₛₖᵢₙ, T_{side}, and Tₜₒₚ for the time t and the heart rate N for the time t into the tensor (step S206 shown in FIG. 21) after the lapse of the time T (YES in step S205 shown in FIG. 21).

Next, the selection unit 46 of the server device 4a selects the learner to be used for estimating the proportional coefficient a from among learner 43-1 to 43-3 prepared in advance. By switching the range of the output layer of the learner according to the environment and the use scene, accurate estimation can be performed. For example, in general, during sleep of a healthy person, the healthy person is in a room and the outside air temperature is also stable. Therefore, the proportional coefficient a is about 2 to 6, and it is sufficient that the number of output layers of the learner is about 40. On the other hand, when a person exercises outdoors, the change of the core body temperature is large in the hot or cold environment, and therefore, it is desirable to widen the range of the output layer of the learner to be prepared.

Therefore, it is desirable to prepare a plurality of learners and switch the learners for each scene in order to improve the accuracy of estimation of the core body temperature. For example, a learner 43-1 corresponding to an exercise state or a hot/cold environment, a learner 43-2 corresponding to a daily life, and a learner 43-3 corresponding to a sleep state are prepared in advance. The selection unit 46 selects a learner to be used by using the temperature Tₛₖᵢₙ of the skin surface, the temperature Tₜₒₚ of the sensor upper part, or the heart rate N as an index.

Specifically, any one case of where the temperature Tₜₒₚ is higher than the threshold value T_{top_th_high}, where the temperature Tₜₒₚ is lower than the threshold value Tt_{op_th_low}, where the temperature Tₛₖᵢₙ is higher than the threshold value T_{skin_th}, or where the heart rate N is higher than the threshold value Nₜₕ is established (YES in step S207 shown in FIG. 21), the selection unit 46 judges that the learner 43-1 is used (step S208 shown in FIG. 21).

T_{top_th_high} is a temperature threshold value for judging whether or not the outside air is in a high hot environment. T_{top_th_low} is a temperature threshold for judging whether or not the outside air is in a low cold environment. T_{skin_th} is a temperature value for judging whether or not the living body 100 is in a heat generation state due to a cold or the like or in a heat generation state due to exercise. Nₜₕ is a heart rate threshold value for judging whether or not the living body 100 is in an exercise state.

On the other hand, when the judgement of the step S207 is not established and the heart rate N is lower than the threshold value N_{th_low} (YES in step S209 shown in FIG. 21), the selection unit 46 judges that the learner 43-3 is used (step S210 shown in FIG. 21). The threshold value N_{th_low} is a heart rate threshold value for judging whether or not the living body 100 is in a sleep state or in a rest state.

The selection unit 46 judges that the learner 43-2 is used (step S211 shown in FIG. 21) when neither of the judgement in the steps S207 and S209 is established (NO in the step S209).

Note that the judgement of steps S207 and S209 may be performed by using the latest values among the respective time series data of the temperatures Tₛₖᵢₙ and Tₜₒₚ for the time T and the heart rate N for the time t, or the judgement may be made using respective representative values (for example, average values) of the temperatures Tₛₖᵢₙ, Tₜₒₚ, and the heart rate N of time t. In addition, when the temperature Tₜₒₚ is measured by a plurality of temperature sensors 11, the judgement of the step S207 may be performed by using the temperature Tₜₒₚ measured by the temperature sensor 11 at one predetermined location, or the judgement may be performed by using a representative value (for example, an average value) of the temperature Tₜₒₚ measured by the plurality of temperature sensors 11.

The learner selected by the selection unit 46 among learners 43-1 to 43-3 of the server device 4a outputs the estimation result of the proportional coefficient a to the tensor inputted from the tensor generation unit 42 (step S212 shown in FIG. 21).

The learners 43-1 to 43-3 need to learn in advance in accordance with the corresponding environment and use scene. That is, the learner 43-1 acquires data of the temperature Tₛₖᵢₙ, T_{side}, Tₜₒₚ, and T_{ref} and the heart rate N of the living body 100 under an exercise state or a hot/cold environment to perform learning. The learner 43-2 acquires data of the temperature Tₛₖᵢₙ, T_{side}, Tₜₒₚ, and T_{ref} and the heart rate N of the living body 100 in a daily life to perform learning. The learner 43-3 acquires data of the temperature Tₛₖᵢₙ, T_{side}, Tₜₒₚ, and T_{ref} and the heart rate N of the living body 100 in a sleep state to perform learning.

The processing of steps S213 to S215 shown in FIG. 21 is the same as the processing of steps S106 to S108 of the first example.

As described above, the core body temperature T_{cbt} is calculated for each time T. When the learners 43-1 to 43-3 are provided on the cloud server side, learning of the learners 43-1 to 43-3 is sequentially advanced, and update and high accuracy can be advanced while using the learners.

Note that in order to protect the temperature sensors 10 and 13, the temperature sensors 10 and 13 may be prevented from directly touching the skin of the living body 100. For example, a thin sheet-like member made of a material having a small heat capacity such as PET resin may be provided on the surface of the housing 17 on the living body side, and the temperature sensors 10 and 13 may measure the temperatures Tₛₖᵢₙ and T_{side} on the skin surface of the living body 100 through the member.

In addition, in the first and second examples, the core body temperature T_{cbt} is calculated after the proportional coefficient a is estimated by the learners 43 and 43-1 to 43-3, but the core body temperature T_{cbt} may be estimated by the learners 43 and 43-1 to 43-3.

When estimating the core body temperature T_{cbt} by the learners 43 and 43-1 to 43-3, the machine learning unit 44 of the server devices 4 and 4a perform machine learning of the learners 43 and 43-1 to 43-3 using the tensor converted from each time series data of the temperatures Tₛₖᵢₙ, T_{side}, Tₜₒₚ, and T_{ref} acquired in advance at the test. Specifically, the machine learning unit 44 sets each time series data of the temperatures Tₛₖᵢₙ, T_{side}, Tₜₒₚ as input variables of the learners 43 and 43-1 to 43-3, sets the core body temperature T_{ref} as output variables of the learners 43 and 43-1 to 43-3, and performs the machine learning of the learners 43 and 43-1 to 43-3 so as to obtain a target output variable.

When the data of the heart rate N is used, the machine learning unit 44 perform machine learning of the learners 43 and 43-1 to 43-3 using the tensor converted from each time series data of the temperatures Tₛₖᵢₙ, T_{side}, Tₜₒₚ, T_{ref}, and the heart rate N acquired in advance at the test. Specifically, the machine learning unit 44 sets each time series data of the temperatures Tₛₖᵢₙ, T_{side}, and Tₜₒₚ and time series data of the heart rate N as input variables of the learners 43 and 43-1 to 43-3, sets the core body temperature T_{ref} as output variables of the learners 43 and 43-1 to 43-3, and performs the machine learning of the learners 43 and 43-1 to 43-3 so as to obtain a target output variable.

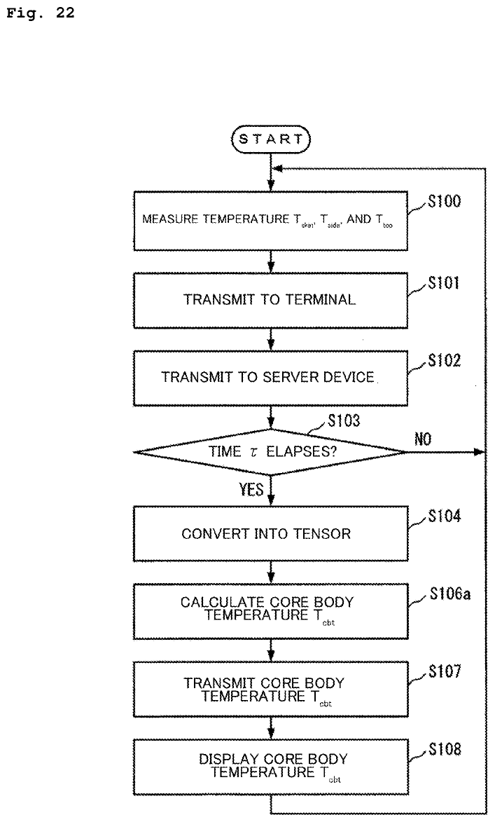

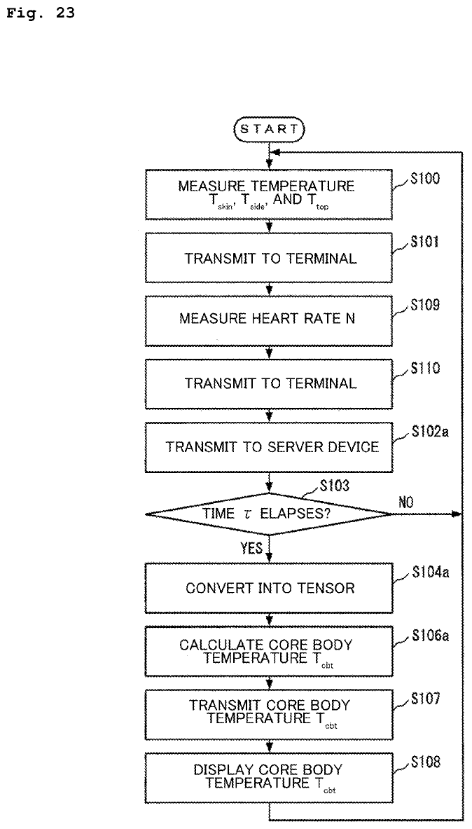

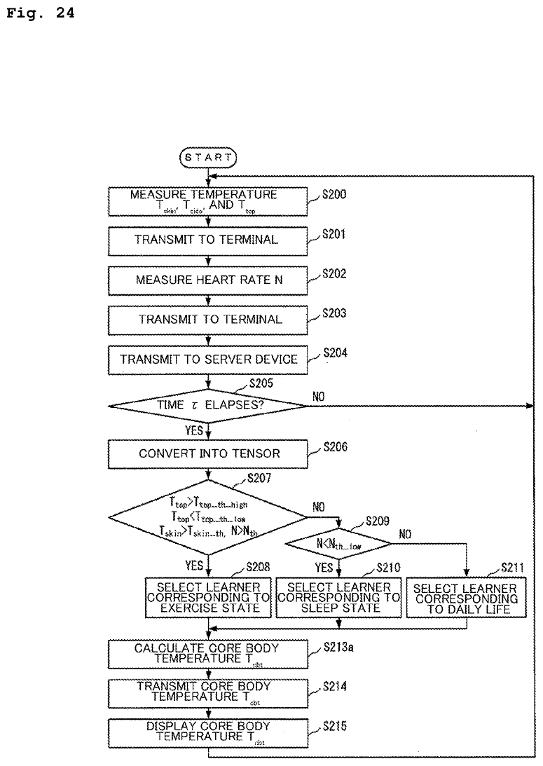

When estimating the core body temperature T_{cbt} by the learners 43 and 43-1 to 43-3, it is needless to say that the temperature calculation unit 45 becomes unnecessary. FIG. 22 and FIG. 23 show the operations of estimating the core body temperature T_{cbt} in the first example, and FIG. 24 shows the operations of estimating the core body temperature T_{cbt} in the second example. FIG. 22 shows an example in which the heart rate measurement unit 2 is not used, and FIG. 23 shows an example in which the heart rate measurement unit 2 is used.

In the examples of FIG. 22 and FIG. 23, the learner 43 outputs the estimation result of the core body temperature T_{cbt} for the tensor inputted from the tensor generation unit 42 (step S106a).

In the example of FIG. 24, the learner selected by the selection unit 46 among the learners 43-1 to 43-3 outputs the estimation result of the core body temperature T_{cbt} with respect to the tensor inputted from the tensor generation unit 42 (step S213a).

Note that since the variation of the core body temperature T_{cbt} is continuous and gentle with respect to the proportional coefficient α, it is generally more accurate to estimate the proportional coefficient α.

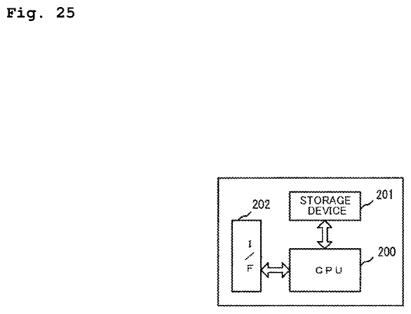

The storage unit 14, the communication unit 15, and the control unit 16 of the temperature measurement unit 1 and 1a to 1g described in the first and second examples can be realized by a computer having a CPU (Central Processing Unit), a storage device, and an interface and a program that controls these hardware resources. FIG. 25 shows a configuration example of the computer.

The computer includes a CPU 200, a storage device 201, and an interface device (I/F) 202. The temperature sensors 10, 11, and 13, the hardware of the communication unit 15, and the like are connected to the I/F 202. In such a computer, a temperature estimation program for realizing the temperature estimation method of the embodiments of present invention is stored in the storage device 201. The CPU 200 executes the processing described in the first and second examples in accordance with the program stored in the storage device 201.

Each of the terminal 3 and the server devices 4, 4a can be realized by the computer.

### Industrial Applicability

Embodiments of the present invention can be applied to techniques for estimating the internal temperature of a test object such as a living body.

### Reference Signs List

1, 1a to 1g Temperature measurement unit
2 Heart rate measurement unit
3 Terminal
4,4a Server device
10, 11, 13 Temperature sensor
12, 20 Heat insulation material
14, 41 Storage unit
15,40 Communication unit
16 Control unit
17, 18 Housing
19 Wiring
21 Internal structure
42 Tensor generation unit
43, 43-1 to 43-3 Learner
44 Machine learning unit

145 Temperature calculation unit
46 Selection unit

1-8. (canceled)

9. A temperature estimation system comprising:
a heat insulation material;
a first temperature sensor on a surface of the heat insulation material, the first temperature sensor facing a test object and configured to measure a temperature of a surface of the test object;
a second temperature sensor configured to measure a temperature inside the heat insulation material immediately above the first temperature sensor;
a third temperature sensor configured to measure a temperature of a surface of the test object at a position remote from the first temperature sensor;
one or more processors; and
a non-transitory computer-readable storage medium storing a program to be executed by the one or more processors, the program including instructions to:
estimate, by a first learning algorithm, a proportional coefficient associated with a thermal resistance of the test object based on a first measurement result of the first temperature sensor, a second measurement result of the second temperature sensor, and a third measurement result of the third temperature sensor; and
calculate an internal temperature of the test object based on the first measurement result of the first temperature sensor, the second measurement result of the second temperature sensor, and the proportional coefficient.

10. The temperature estimation system according to claim 9, further comprising:
a heart rate measurement device configured to measure a heart rate of the test object, wherein the instructions to estimate the proportional coefficient includes further instructions to estimate the proportional coefficient based on the first measurement result of the first temperature sensor, the second measurement result of the second temperature sensor, the third measurement result of the third temperature sensor, and a fourth measurement result of the heart rate measurement device.

11. The temperature estimation system according to claim 10, wherein the program includes further instructions to:
prepare a plurality of learning algorithms in advance for coping with a state of the test object or an environment around the test object; and
select the first learning algorithm from the plurality of learning algorithms corresponding to the state of the test object or the environment around the test object based on the first measurement result of the first temperature sensor, the second measurement result of the second temperature sensor, or the fourth measurement result of the heart rate measurement device.

12. The temperature estimation system according to claim 9, wherein the program includes further instructions to:
prepare a plurality of learning algorithms in advance for coping with a state of the test object or an environment around the test object; and
select the first learning algorithm from the plurality of learning algorithms corresponding to the state of the test object or the environment around the test object based on the first measurement result of the first temperature sensor or the second measurement result of the second temperature sensor.

13. A temperature estimation system comprising:
a heat insulation material;
a first temperature sensor on a surface of the heat insulation material, the first temperature sensor facing a test object and configured to measure a temperature of a surface of the test object;
a second temperature sensor configured to measure a temperature inside the heat insulation material immediately above the first temperature sensor;
a third temperature sensor configured to measure a temperature of a surface of the test object at a position remote from the first temperature sensor;
one or more processors; and
a non-transitory computer-readable storage medium storing a program to be executed by the one or more processors, the program including instructions to:
estimate, by a first learning algorithm, an internal temperature of the test object based on a first measurement result of the first temperature sensor, a second measurement result of the second temperature sensor, and a third measurement result of the third temperature sensor.

14. The temperature estimation system according to claim 13, further comprising:
a heart rate measurement device configured to measure a heart rate of the test object, wherein the instructions to estimate the internal temperature of the test object further instructions to estimate internal temperature of the test object based on the first measurement result of the first temperature sensor, the second measurement result of the second temperature sensor, the third measurement result of the third temperature sensor, and a fourth measurement result of the heart rate measurement device.

15. The temperature estimation system according to claim 14, wherein the program includes further instructions to:
prepare a plurality of learning algorithms in advance for coping with a state of the test object or an environment around the test object; and
select the first learning algorithm from the plurality of learning algorithms corresponding to the state of the test object or the environment around the test object based on the first measurement result of the first temperature sensor, the second measurement result of the second temperature sensor, or the fourth measurement result of the heart rate measurement device.

16. The temperature estimation system according to claim 13, wherein the program includes further instructions to:
prepare a plurality of learning algorithms in advance for coping with a state of the test object or an environment around the test object; and
select the first learning algorithm from the plurality of learning algorithms corresponding to the state of the test object or the environment around the test object based on the first measurement result of the first temperature sensor or the second measurement result of the second temperature sensor.

17. A temperature estimation method comprising:
a first step of measuring a first temperature of a surface of a test object by a first temperature sensor provided on a surface of a heat insulation material facing the test object;
a second step of measuring a second temperature inside the heat insulation material immediately above the first temperature sensor by a second temperature sensor;
a third step of measuring a third temperature of a surface of the test object remote from the first temperature sensor by a third temperature sensor;
a fourth step of estimating a proportional coefficient associated with a thermal resistance of the test object by a first learning algorithm based on the first temperature, the second temperature, and the third temperature; and
a fifth step of calculating an internal temperature of the test object based on the first temperature, the second temperature, and the proportional coefficient.

18. The temperature estimation method according to claim 17, further comprising:
a sixth step of measuring a heart rate of the test object, wherein the fourth step comprises a step of estimating the proportional coefficient based on the first temperature, the second temperature, the third temperature, and the heart rate of the test object.

19. The temperature estimation method according to claim 18, further comprising:
a seventh step of preparing a plurality of learning algorithms in advance for coping with a state of the test object or an environment around the test object; and
an eighth step of selecting the first learning algorithm from the plurality of learning algorithms corresponding to the state of the test object or the environment around the test object based on the first temperature, the second temperature, or the heart rate of the test object.

20. The temperature estimation method according to claim 17, further comprising:
a seventh step of preparing a plurality of learning algorithms in advance for coping with a state of the test object or an environment around the test object; and
an eighth step of selecting the first learning algorithm from the plurality of learning algorithms corresponding to the state of the test object or the environment around the test object based on the first temperature or the second temperature.
